(19) United States
(12) Patent Application Publication
(10) Pub. No.: US 2024/0016406 A1
(43) Pub. Date: Jan. 18, 2024

(54) LED LIGHT PATCH, SKIN CARE DIAGNOSTIC SYSTEM USING THE SAME, AND METHOD FOR OPERATING THE SAME

(71) Applicant: KOREA PHOTONICS TECHNOLOGY INSTITUTE, Gwangju (KR)

(72) Inventors: Gi Dong SA, Gwangju (KR); Ja Yeon KIM, Gwangju (KR); Sa Ung KIM, Gwangju (KR); Ji Ho JEONG, Jeonju-si, Jeollabuk-do (KR); Eunbee KIM, Gwangju (KR)

(73) Assignee: KOREA PHOTONICS TECHNOLOGY INSTITUTE, Gwangju (KR)

(21) Appl. No.: 18/253,072

(22) PCT Filed: Feb. 16, 2021
(86) PCT No.: PCT/KR2021/001938

§ 371 Date: May 16, 2023

(30) Foreign Application Priority Data
Nov. 24, 2020 (KR) 10-2020-0158922

(51) Int. Cl. A61B 5/0537; A61B 5/00

(52) U.S. Cl. CPC A61B 5/0537; A61B 5/0077; A61N 5/0616

(57) ABSTRACT

The present invention provides a skin care diagnostic system characterized in that an LED light path is linked to a user terminal device having: a camera module for capturing an image of a skin surface; and a skin care service application generating control solution information of the LED light patch by analyzing the image captured by the camera module and an impedance measurement value received from the LED light patch. The LED light patch comprises: a BIA module for sensing the bioelectrical impedance of the skin surface; a BLE module for transmitting the impedance measurement value measured by the BIA module and receiving the control solution information of the LED light patch; a battery module comprising a thin-film battery; an LED module on which micro-LED devices are mounted in an array; and a control processor performing area-specific control, dimming control, wavelength control and user-selected care time control of the LED module in accordance with the received control solution information.

### TECHNICAL FIELD

The present invention relates to an LED light patch and a skin diagnostic technology using the same, more specifically, to an LED light patch, a skin care diagnostic system using the same, and a method for operating the same that are capable of performing skin care through artificial intelligence-based diagnosis obtained with impedance measurement information by facial area and images captured by facial area.

### BACKGROUND ART

As a home beauty device market has been drastically grown domestically and internationally, recently, people's attention is drawn to LED masks in the market, but safety problems of the LED masks and the reliable proof of their skin improvement effect still cause controversy.

As trends in which emphasis is put on appearance and cost effectiveness are spread, further, ‘home care’, through which a user takes care of his or her body or health for himself or herself, becomes very popular. Accordingly, skin care products using light, fine current, ultrasonic light, high frequency, and the like are newly introduced in a domestic skin cosmetic device market.

Accordingly, advantages LEDs have had, such as selective wavelength function utilization, high freedom of size adjustment, flexible characteristics, and new reaction effects by wavelength, have been recently revealed through numerous studies and papers, and through the utilization of such advantages, there are tries to newly develop various products such as a low-power flexible display product using micro-LEDs and an external attachment skin treatment product.

As one of the technologies related to such tries, a skin attachment cosmetic patch through wireless communication control is disclosed in Korean Utility Model Application Laid-open No. 20-2019-0002254 (Dated Sep. 5, 2019), and the skin attachment cosmetic patch includes: a circuit board; a plurality of light emitting diodes (LEDs) arranged on top of the circuit board; an electrode printing film laid horizontally on the LEDs; an electrode located on the electrode printing film; and a battery for supplying electricity to the circuit board, whereby any one or more of LED light sources and fine current are supplied to skin.

### DISCLOSURE

### Technical Problem

Accordingly, the present invention has been made in view of the above-mentioned problems occurring in the related art, and it is an object of the present invention to provide an LED light patch that is capable of performing power supply stably and keeping the bonded states of circuits even if varied in shape and being applied to various skin areas of a human body.

It is another object of the present invention to provide an LED light patch and a skin care diagnostic system using the same that are capable of providing skin care diagnosis solutions including area-specific control, dimming control, wavelength control, and user-selected care time control of an LED module in accordance with the skin moisture measurement information obtained by the LED light patch and the skin diagnosis image information obtained by a camera of a smartphone.

It is yet another object of the present invention to provide a customized skin care diagnostic system and a method for operating the same that are capable of reflecting the images by skin area captured by a camera module of a smartphone, the bioelectrical signal values received from LED light patches, and data of skin conditions of users by age, sex, job, and country to thus perform artificial intelligence-based data analysis and diagnosis.

### Technical Solution

To accomplish the above-mentioned objects, according to an aspect of the present invention, a light emitting diode (LED) light patch may include: a Bioelectrical Impedance Analysis (BIA) module for sensing the bioelectrical impedance generated by flowing fine current to a stretchable circuit board varied in shape according to contraction and relaxation to thus measure moisture content of a skin surface; a Bluetooth Low Energy (BLE) module for transmitting the impedance measurement value measured by the BIA module to a user terminal device and receiving the control solution information generated from a skin care service application of the user terminal device; a battery module having a flexible thin-film battery; an LED module on which micro-LEDs are mounted in an array; a power supply circuit having a regulator circuit adapted to supply a given voltage to the respective modules connected to the battery module, a booster circuit adapted to boost the voltage by dimming level, and a battery protection circuit; and a control processor for performing area-specific control, dimming control, wavelength control and user-selected care time control of the LED module in accordance with the received control solution information, whereby even if the LED light patch is varied in shape, the power supply state and the bonded states of the circuits are kept stably.

Further, the LED light patch according to the present invention is made customizedly and applied to a specific area of facial skin or a skin area of a body and controlled by the skin care service application of the user terminal device.

To accomplish the above-mentioned objects, according to another aspect of the present invention, a skin care diagnostic system may include: LED light patches each having a BIA module for sensing the bioelectrical impedance to measure moisture content of a skin surface, a BLE module for transmitting the impedance measurement value measured by the BIA module to a user terminal device and receiving the control solution information of the corresponding LED light patch generated from the user terminal device, a battery module having the form of a flexible thin-film battery, an LED module on which micro-LED devices are mounted in an array, and a control processor performing area-specific control, dimming control, wavelength control and user-selected care time control of the LED module in accordance with the received control solution information; and the user terminal device having a camera module for capturing an image of the skin surface and a skin care service application for diagnosing the images captured by the camera module and the impedance measurement values received from the LED light patches to generate the control solution information of the LED light patches, while interworking with the LED light patches.

According to the present invention, further, the LED light patches may be made customizedly with 8 areas of the facial skin that are made by dividing the entire facial area into a forehead area, both areas around eyes, both cheek areas, both nasolabial fold areas around nose and mouth, and a neck area, while being controlled by the skin care service application of the user terminal device, through independent signals.

According to the present invention, further, the skin care service application may diagnose skin wrinkle and whitening conditions with levels 1 to 5 and an off mode through the image of the specific area captured by the camera module and diagnoses skin moisture content with grades 1 to 5 and an off mode through the impedance measurement value sensed by the BIA module, so that the skin care service application generates the control solution information for controlling the LED module attached to the specific area.

According to the present invention, further, the skin care service application may interwork with a skin care management server to collect and store the captured skin image and the skin moisture content and thus generate the control solution information for controlling the LED module, while reflecting data of skin conditions of users by age, job, sex, and country.

According to the present invention, further, the control solution information may include control information by area for 8 areas of the face, three areas of the forehead, and three areas of the neck, dimming control information for brightness control by diagnosis level, wavelength control information for 630 nm wavelength for wrinkle care and for both of 630 nm and 850 nm wavelengths for whitening care, and care time control information as a user option.

To accomplish the above-mentioned objects, according to yet another aspect of the present invention, a method for operating one or more LED light patches and a skin care diagnostic system using the LED light patches may include the steps of: executing a skin care service application embedded in a smartphone to perform Bluetooth interworking with the LED light patches; capturing images of specific facial areas through a camera module of the smartphone to check the wrinkle and whitening conditions of the specific facial areas; attaching the LED light patches to the specific facial areas, measuring impedance values of the specific facial areas to check skin moisture content, and transmitting the impedance values to the skin care service application; analyzing and diagnosing skin conditions through the skin care service application, based on the image and impedance information of the specific facial areas; generating control solution information of the LED light patches, based on the skin condition diagnosis elements of the specific facial areas analyzed by the skin care service application; and allowing the LED light patches to receive the control solution information to irradiate light onto the specific facial areas.

According to the present invention, further, the step of analyzing and diagnosing skin conditions may be performed by allowing the skin care service application to interwork with a skin care management server to collect and store the captured skin images and the skin moisture content and thus generate the control solution information for controlling the LED modules, while reflecting data of skin conditions of users by age, job, sex, and country.

According to the present invention, further, the specific facial areas to which the LED light patches are attached may be any one or more areas of a forehead area, both areas around eyes, both cheek areas, both nasolabial fold areas around nose and mouth, and a neck area, and the LED patches attached to the specific facial areas may be controlled by the skin care service application through independent signals.

According to the present invention, further, the control solution information may include control information by area for 8 areas of the face, three areas of the forehead, and three areas of the neck, dimming control information for brightness control by diagnosis level, and wavelength control information for 630 nm wavelength for wrinkle care and for both of 630 nm and 850 nm wavelengths for whitening care.

According to the present invention, the control solution information may further include care time control information as a user option.

### Advantageous Effects

According to the present invention, the LED light patches, the skin care diagnostic system using the LED light patches, and the method for operating the skin care diagnostic system are applied to skin care markets and bio-healthcare industry with the adoption of wearable devices, thereby expecting the creation of a new fusion market.

According to the present invention, further, the stretchable LED light patches are customized according to the free skin areas as well the face of the user and thus configured to allow the LED modules to be controlled according to the artificial intelligence-based control solution information through the simple control of the user terminal device such as the smartphone, thereby maintaining the skin moisture content and performing the skin care for skin wrinkle and whitening effectively.

### BRIEF DESCRIPTION OF DRAWINGS

FIG. 1 is a block diagram showing a skin care diagnostic system according to the present invention.

FIG. 2 is a block diagram showing an LED light patch according to the present invention.

FIG. 3 is an exemplary view showing face attachment areas for the LED light patches of the present invention independently applied to a facial skin surface.

FIG. 4 is a flowchart showing a method for operating a skin care diagnostic system using LED light patches according to the present invention.

FIG. 5 is a block diagram showing data and control signals of the skin care diagnostic system using a smartphone and the LED light patches according to the present invention.

FIG. 6 is an exemplary view showing a mapping table of control solution information generated according to skin condition diagnosis elements according to the present invention.

FIG. 7 is a flowchart showing Bluetooth signal transmission between the LED light patches and the smartphone according to the present invention.

FIG. 8 is an exemplary view showing transmission data frames for Bluetooth signal transmission bit allocation examples for wrinkle and whitening control solution information according to the present invention.

FIG. 9 is an exemplary view showing transmission data frames for Bluetooth signal transmission bit allocation examples for whitening care-dedicated control solution information according to the present invention.

### BEST MODE FOR INVENTION

According to one aspect of the present invention, an LED light patch includes a Bioelectrical Impedance Analysis (BIA) module for sensing the bioelectrical impedance generated by flowing fine current to a stretchable circuit board varied in shape according to contraction and relaxation to thus measure moisture content of a skin surface; a Bluetooth Low Energy (BLE) module for transmitting the impedance measurement value measured by the BIA module to a user terminal device and receiving the control solution information generated from a skin care service application of the user terminal device; a battery module having a flexible thin-film battery; an LED module on which micro-LED devices are mounted in an array; a power supply circuit having a regulator circuit adapted to supply a given voltage to the respective modules connected to the battery module, a booster circuit adapted to boost the voltage by dimming level, and a battery protection circuit; and a control processor for performing area-specific control, dimming control, wavelength control and user-selected care time control of the LED module in accordance with the received control solution information, whereby even if the LED light patch is varied in shape, the power supply state and the bonded states of the circuits are kept stably.

### MODE FOR INVENTION

The terms used in the specification and claims of the present invention are to be given their ordinary and customary meaning to a person of ordinary skill in the art and are not to be limited to a special or customized meaning unless expressly so defined herein. It should be noted that the use of particular terms when describing certain features or aspect of the disclosure should not be taken to imply that the term is being re-defined herein to be restricted to include any specific characteristics of the features or aspects of the disclosure with which that term is associated.

Therefore, an embodiment of the present invention and a configuration in the drawings are only the most desirable embodiment of the present invention, which does not limit the technical scope of the present invention. Accordingly, it should be appreciated that the present invention covers all the modifications, equivalents, and replacements within the idea and technical scope of the present invention.

Hereinafter, preferred embodiments of the present invention will be described in detail with reference to the accompanying drawings.

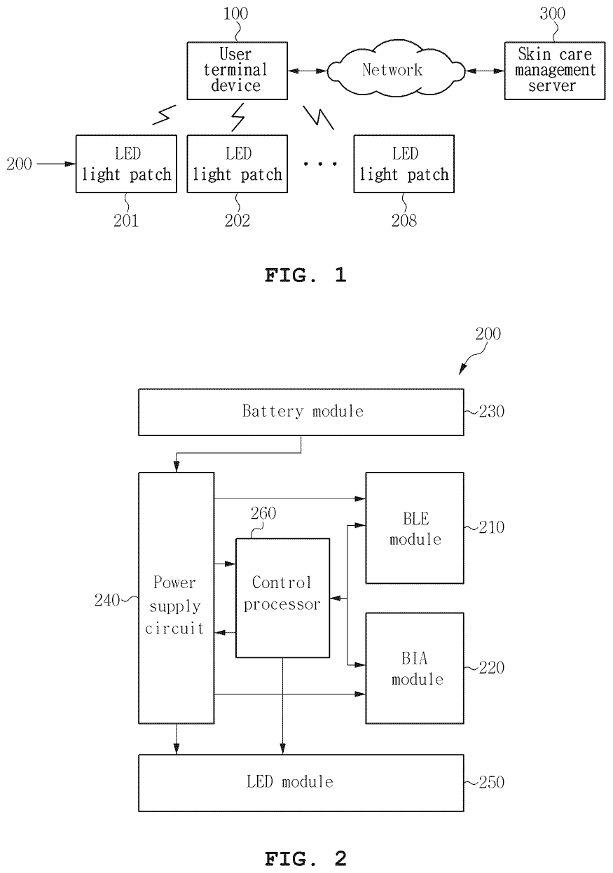

FIG. 1 is a block diagram showing a skin care diagnostic system according to the present invention.

As shown, a skin care diagnostic system according to the present invention includes a user terminal device 100, one or more LED light patches 200 attached to a user's facial skin areas, and a skin care management server 300 interworking with the user terminal device 100.

The user terminal device 100 generally represents a smartphone, but it may include a smart device or computing device corresponding to the smartphone, without being limited to the smartphone. In the description, an explanation of the smartphone as the user terminal device will be given.

The smartphone 100 includes a camera module for capturing an image of a skin surface, a Bluetooth module for wireless connection with the camera module and the LED light patches 200, and a skin care service application (hereinafter, referred to as app) installed by a processor embedded therein.

Further, the smartphone 100 interworks with the skin care management server 300 through a communication module connected to an external network.

The skin care management server 300 provides or distributes the skin care service app to the user of the LED light patches for the management of the LED light patch user and further includes a database adapted to collect and store the images of the user's skin surface and his or her skin moisture content and to further collect and store data of skin conditions of users by age, job, sex, and country to allow the skin care service app to utilize the stored images and data.

The skin care service app is a skin care solution providing program for controlling the LED light patches that generates skin care solution information through diagnosis made by artificial intelligence-based composite data analysis based on the images by skin area captured by the camera module of the smartphone and the bioelectrical signal values received from the LED light patches.

Further, the skin care management server receives the user's skin images captured by the smartphone and the bioelectrical signal measurement values for measuring the skin moisture content, produces the skin care solution information through the diagnosis made by the artificial intelligence-based composite data analysis, and thus provides the skin care solution information to the skin care service app.

Accordingly, the LED light patches 200 of the present invention transmit the bioelectrical impedance measurement values for measuring the moisture content of the skin surface to the smartphone, and the smartphone receives the skin care solution information through the skin care service app to control the LED light patches 200, so that the LED light patches 200 are customized AI-based active skin patches that perform the user's skin care.

In this case, the skin care solution information includes LED wavelengths, outputs, and time control information for light irradiation of the LED light patches 200.

FIG. 2 is a block diagram showing the LED light patch according to the present invention, and as shown, the LED light patch 200 according to the present invention includes a BLE module 210, a BIA module 220, a battery module 230, a power supply circuit 240, an LED module 250, and a control processor 260.

The BLE module 210 is a Bluetooth circuit having a Bluetooth chip, and the Bluetooth chip is a system on chip that includes a Bluetooth low energy (BLE) protocol stack allowing BLD communication and performs interfacing with wide-range sensors.

Accordingly, the BLE module 210 is provided with a Bluetooth antenna circuit, and the antenna circuit is embedded in the circuit board of the LED light patch. For example, if a general Bluetooth antenna has a thickness of 2 mm, the antenna is embedded in the circuit board within the thickness of the circuit board, so that the thickness of the LED light patch can be more reduced.

As a result, the LED light patch 200 transmits a BIA sensor measurement value to the skin care service app of the smartphone through Bluetooth communication or receives the control solution information signals of the LED module 250 generated through diagnosis.

The BIA module 220 includes a BIA sensor for sensing an impedance index generated by flowing fine current to the skin surface to measure skin moisture content, under the principle in which a degree of electricity passing, that is, a resistance, is varied according to moisture content, and a sensing electrode (BIA tip) for flowing the fine current to the skin surface to sense the impedance index.

In this case, the BIA module 220 transmits the skin moisture measurement data to the smartphone or the external communication device corresponding to the smartphone through the BLE module 210.

The battery module 230 is a battery for supplying power to the LED light patch 200, particularly a thin-film battery that is flexible so that it can be applied to a curved surface with a given radius of a curve such as a facial surface. For example, the battery module 230 is a flexible thin-film battery having a thickness of 0.3 to 0.4 mm.

In this case, the battery module is configured to allow a single thin-film battery to be laid on the circuit board or to allow a given number of small thin-film batteries to be arranged vertically and horizontally in an array and laid on the circuit board.

The power supply circuit 240 includes a regulator circuit adapted to supply a given voltage to the respective modules connected to the battery module, a booster circuit adapted to boost the voltage supplied from the battery by dimming level according to brightness outputs, and a battery protection circuit for preventing the battery from being overcharged or overdischarged.

Further, the power supply circuit 240 includes a digital-to-analog converter (DAC) for receiving a level control signal for dimming control of the LED module from the control processor 260 to control the brightness of the LED module.

Accordingly, the booster circuit boosts the voltage by dimming level by means of the level control signal received from the control processor 260 and controls the brightness of the LED module.

The LED module 250 is configured to allow micro-LEDs as light sources with a size of a hair thickness to be arranged in an array having given numbers of rows and columns on the stretchable circuit board of the LED light patch 200 and then mounted on the circuit board through transferring and bonding. Generally, an LED with the size of 10 to 100 um is called a micro-LED. Further, the LEDs support the wavelengths of 630 nm and 850 nm. In this case, the LED having the wavelength of 630 nm operates for wrinkle care, and the LED having the wavelength of 850 nm operates for whitening care.

The control processor 260 is a micro controller processor that analyzes the LED control signal of the skin care control solution information received from the BLE module and thus performs the area-specific control, dimming control, wavelength control and user-selected care time control of the LED module according to the analyzed result.

Further, the control processor 260 provides the signal transmission and reception control of the BLE module 210, the operation control of the BIA module 220, and battery level measurement and notification.

As mentioned above, the LED light patch 200 according to the present invention is a stretchable patch that makes use of the flexible printed circuit board (FPCB) that is freely bent or extended or contracted so that even if it is varied in shape, the power supply state and the bonded states of the circuits can be kept stably. Accordingly, even though the modules and the electronic circuits mounted on the LED light patch 200 are varied in shape, they can keep the power supply state and the bonded states stably.

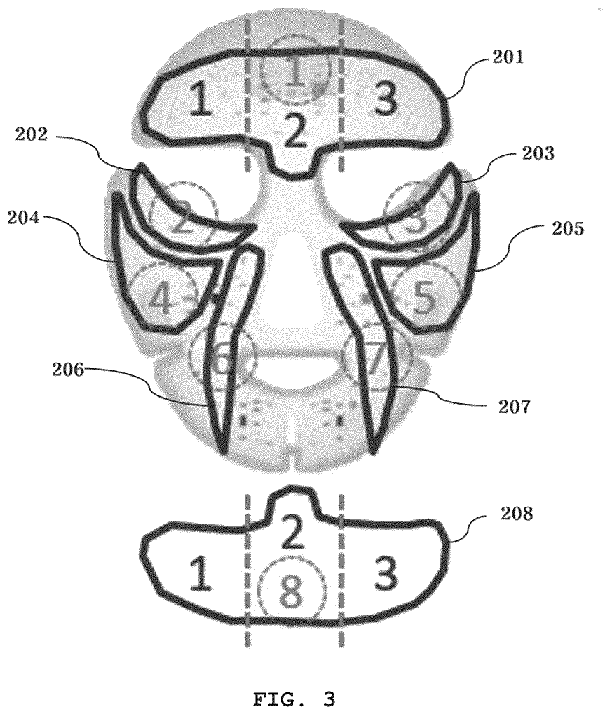

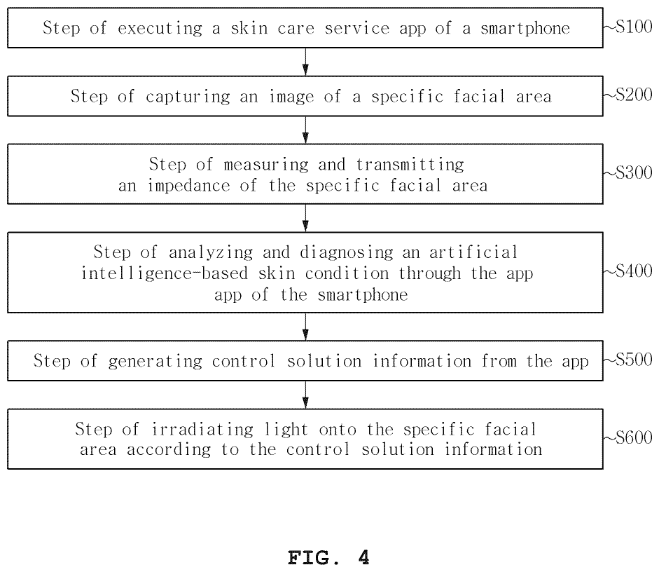

FIG. 3 is an exemplary view showing attachment areas of the LED light patches according to the present invention, which are independently applied to the facial skin surface.

As shown in FIG. 3, the LED light patches according to the present invention are made customizedly with 8 areas of the facial skin that are defined by dividing the entire facial area into a forehead area 201, both areas around eyes 202 and 203, both cheek areas 204 and 205, both nasolabial fold areas 206 and 207 around nose and mouth, and a neck area 208. Accordingly, the LED light patches are attached to the respective areas, independently of one another.

Further, the LED light patches 200 may be made customizedly with other skin areas except for the 8 areas of the facial skin, and therefore, it can be obvious that the LED light patches 200 are applied to the skin areas, independently of one another.

In this case, the LED light patches for the left and right cheek areas 204 and 205 are provided only for whitening care. This is because wrinkles are not generated on the cheeks. The LED light patches 201, 202, 203, 206, 207, and 208 excepting the left and right cheek patches 204 and 205 are provided for both of wrinkle care and whitening care, but of course, they may not be limited thereto.

The LED light patches 201 and 208, which are attached to the forehead and the neck, are subdivided into left, right, and middle areas 1, 2 and 3 of the forehead and left, right, and middle areas 1, 2 and 3 of the neck, and the sensing electrodes (BIA tips) of the BIA modules for impedance measurement of the subdivided areas are located on the subdivided areas.

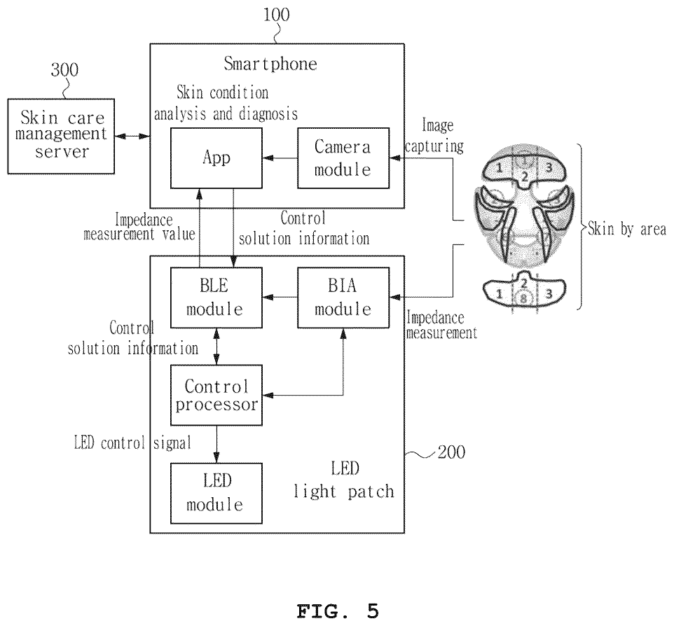

FIG. 4 is a flowchart showing a method for operating a skin care diagnostic system using the LED light patch according to the present invention, and FIG. 5 is a block diagram showing data and control signals of the skin care diagnostic system using the smartphone and the LED light patches according to the present invention.

As shown, a method for operating a skin care diagnostic system using a LED light patch according to the present invention includes the steps of executing a skin care service app of a smartphone (S100), capturing an image of a specific facial area through a camera module of the smartphone (S200), measuring and transmitting an impedance of the specific facial area through a BIA module of the LED light patch (S300), analyzing and diagnosing a skin condition through the skin care service app of the smartphone (S400), generating control solution information from the skin care service app (S500), and irradiating light onto the specific facial area according to the control solution information (S600).

The step (S100) of executing the skin care service app of the smartphone is the step in which the skin care service app installed on the smartphone is executed to operate Bluetooth pairing of the smartphone and the LED light patch and thus prepare skin diagnosis.

The step (S200) of capturing the image is the step in which the image for checking wrinkle and whitening conditions of the specific facial area is acquired by the camera module of the smartphone.

The step (S300) of measuring and transmitting the impedance is the step in which so as to check the skin moisture content of the specific facial area to which the LED light patch is attached, a BIA module flows a current to the skin surface of the specific facial area according to a moisture content information request of the skin care service app, senses the impedance index, and thus transmits the measured impedance index to the skin care service app of the smartphone through a BLE module.

The step (S400) of analyzing and diagnosing the skin condition is the step in which skin condition analysis elements such as wrinkles, whitening and moisture content are analyzed and diagnosed on the basis of the acquired image for the specific facial area and the impedance index information, and in addition thereto, artificial intelligence-based data analysis and diagnosis is made with data of skin conditions by age, job, sex, and country.

According to the present invention, the skin wrinkle condition diagnosis through the captured image is made with five-level diagnosis elements. The wrinkle condition diagnosis elements include weak wrinkle, weak and mild wrinkle, mild wrinkle, mild and strong wrinkle, and strong wrinkle, and further, they include no care condition in which no care is needed because of no wrinkle. In this case, the weak wrinkle means that weak wrinkles exist between the eyebrows to cause light care.

So as to make a skin whitening condition diagnosis for the captured image, further, given unit brightness is set and divided into five-level diagnosis elements. As the five-level diagnosis elements, skin brightness is divided into darkness grades 1 to 5, and if the skin brightness is greater than given brightness, a skin brightness grade on which whitening care is not required is further included in the five-level diagnosis elements. In this case, the darkness grade 3 means that appropriate whitening care is needed.

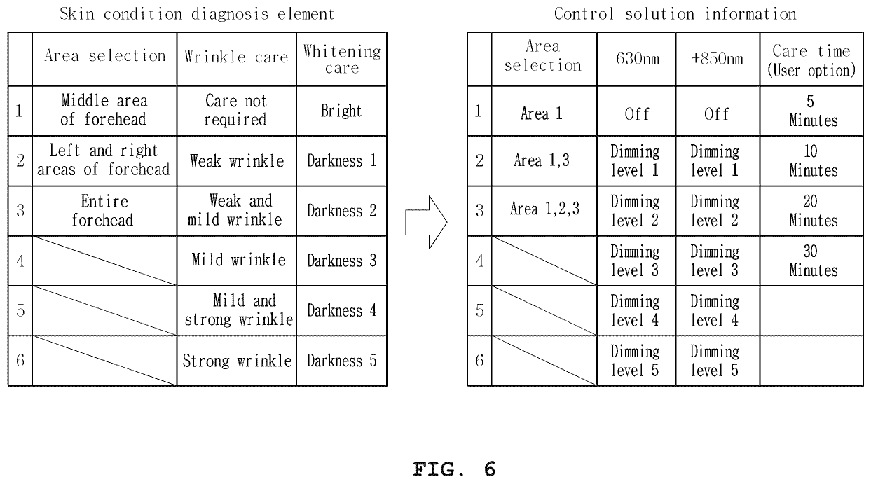

Referring to the skin condition diagnosis elements of FIG. 6 according to the present invention, the number of cases of expectation diagnosis through the AI-customized analysis for the forehead include three forehead areas (the forehead middle area (the area 2), the forehead left and right areas (the areas 1 and 3), and the entire forehead area), the six wrinkle care diagnosis elements on the forehead (the levels 1 to 5+the level in which care is unnecessary), and the six whitening care diagnosis elements on the forehead (the grades 1 to 5+bright skin level), and accordingly, total 108 of cases of condition diagnosis elements are generated. It can be appreciated from the foregoing that total 108 of cases of condition diagnosis elements may be generated on the neck area.

Further, five-level skin moisture diagnosis elements are provided to perform the skin moisture diagnosis through the BIA module and include very dry, dry, slightly dry, mild, and slightly wet skin.

FIG. 6 is an exemplary view showing a mapping table of the control solution information generated according to the skin condition diagnosis elements according to the present invention.

In the control solution information of the wrinkle condition diagnosis, the whitening condition diagnosis and the skin moisture condition diagnosis, the five levels and grades are dividedly provided, but they are just exemplary in the embodiment of the present invention. Therefore, they will be arbitrarily adjusted according to embodiments of the present invention.

The step (S500) for generating the control solution information is the step in which the control solution information of the LED light patch is generated from the skin care service app through the mapping table of FIG. 6, based on the analyzed diagnosis elements for the skin conditions by facial area.

According to an embodiment of the present invention, the middle portion of the forehead as the area 2 is selected, and as the results of the skin image information and the impedance measurement information, it is assumed that moisture content is mild, the wrinkle care diagnosis element is weak wrinkle, and skin brightness as the whitening care diagnosis element is bright skin grade requiring no whitening care. If the diagnosis elements match the control solution information, it can be appreciated that the control solution information of the area 2 is generated to perform a dimming level 1 for the wavelength of 630 nm and to operate an off mode for the wavelength of 850 nm.

According to another embodiment of the present invention, the entire area of the forehead as the areas 1, 2, and 3 is selected, and as the results of the skin image information and the impedance measurement information, it is assumed that moisture content is slightly wet, the wrinkle care diagnosis element is wrinkle care not required, and skin brightness as the whitening care diagnosis element is darkness grade 3 requiring appropriate whitening care. If the diagnosis elements match the control solution information, it can be appreciated that the control solution information of the areas 1, 2, and 3 is generated to perform a dimming level 3 for the wavelengths of 630 nm and 850 nm.

According to yet another embodiment of the present invention, the left and right areas of the forehead as the areas 1 and 3 are selected, and as the results of the skin image information and the impedance measurement information, it is assumed that moisture content is dry, the wrinkle care diagnosis element is strong wrinkle requiring wrinkle care, and skin brightness as the whitening care diagnosis element is darkness grade 5 requiring strong whitening care. If the diagnosis elements match the control solution information, it can be appreciated that the control solution information of the areas 1 and 3 is generated to perform a dimming level 5 for the wavelengths of 630 nm and 850 nm.

In this case, at the step (S500) for generating the control solution information, care time as a user option is divided by level and added to the control solution information. For example, the control solution information is set so that the care time has the units of 5, 10, 20, and 30 minutes and is thus adjusted through dimming levels 1, 2, 3 and 4.

The step (S600) for irradiating light is the step in which the control solution information generated through the skin care service app is received and according to the received information, the LED module of the LED light patch attached to the facial skin is controlled to irradiate light to the facial skin. In specific, the control solution information includes area control, dimming control, wavelength control, and care time levels as the user option, and through the control solution information, the operation of the LED light patch is controlled.

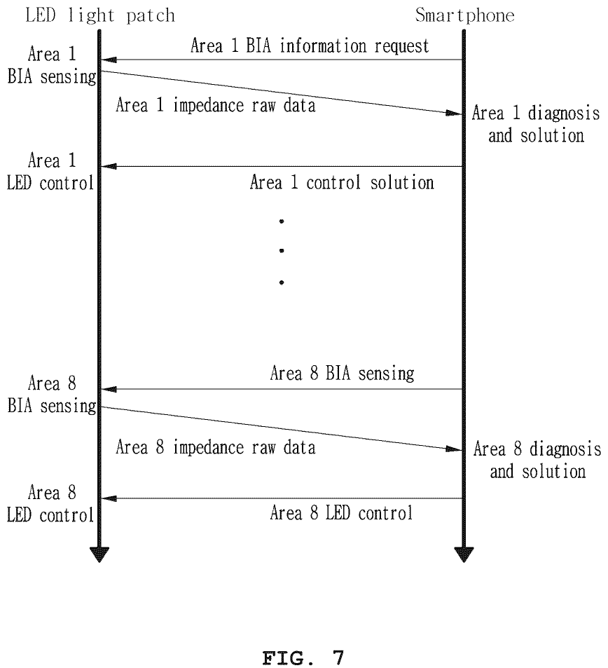

FIG. 7 is a flowchart showing Bluetooth signal transmission between the LED light patches and the smartphone according to the present invention, and in this case, Bluetooth signal transmission is shown after the image capturing for the facial skin surface.

In specific, the LED light patches according to the present invention are attached, independently of one another, to a forehead area (area 1), both areas around eyes (areas 2 and 3), both cheek areas (areas 4 and 5), both nasolabial fold areas (areas 6 and 7) around nose and mouth, and a neck area (area 8) on the facial skin surface, and they have Bluetooth pairing with the smartphone.

The LED light patches receive the moisture condition information request signals by area sequentially from the skin care service app of the smartphone.

After that, each LED light patch allows the BIA module to flow a current to the skin surface of the area requested and thus measure an impedance index. The measured impedance index as raw data is transmitted to the skin care service app.

Next, the skin care service app, which receives the impedance index, transmits the control solution information generated through the skin condition diagnosis for the area requested to the corresponding LED light patch.

After that, the LED light patch, which receives the control solution information, performs the skin care for the area requested through the LED control, based on the control solution information of the area requested.

In specific, as shown in FIG. 7, the skin care for the areas 1 to 8 to which the LED light patches are attached is performed through the sequential LED control according to Bluetooth communication.

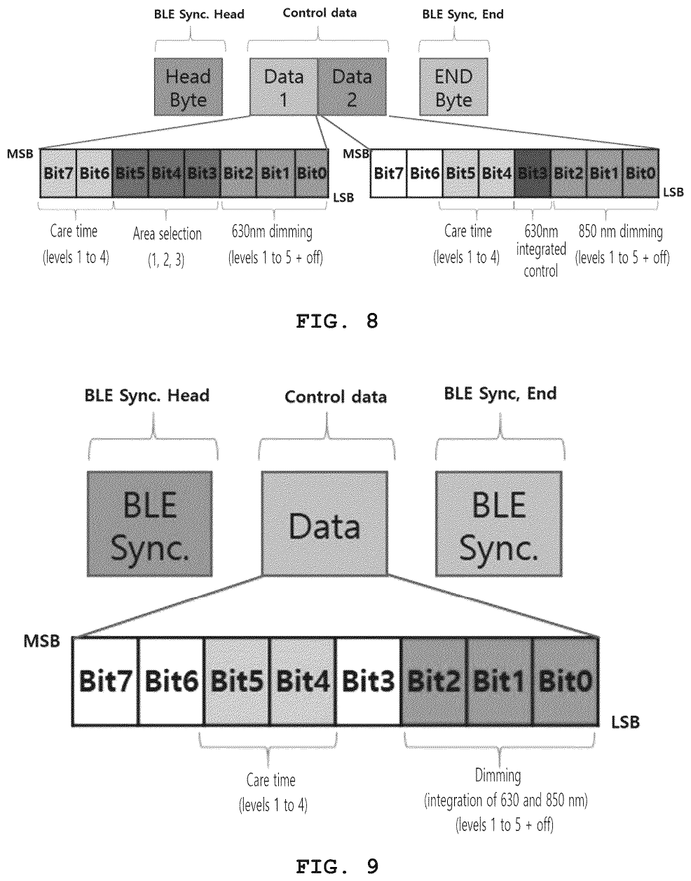

Further, FIG. 8 is an exemplary view showing transmission data frames for Bluetooth signal transmission bit allocation examples for wrinkle and whitening control solution information according to the present invention.

Two data bytes are provided as transmission data frames by area for wrinkle and whitening care. In specific, data 1 includes the control solution information for wrinkle care, and 1 byte of data 2 is assigned to have the control solution information for whitening care.

In the data 1, two bits as first bits of most significant bits (MSB) are assigned as bits for care time values (levels 1 to 4), next three bits as bits for area selection, and last three bits as information bits for six cases including dimming levels 1 to 5 for wrinkle care and for off mode selection.

Further, in the data 2, two bits as first bits of MSB are assigned as bits for reserved areas, next two bits as bits for care time values (levels 1 to 4), subsequent three bits as bits for integrated control for 630 nm LEDs for whitening care, and last three bits as information bits for six cases including dimming levels 1 to 5 for 850 nm LEDs and for off mode selection.

FIG. 9 is an exemplary view showing transmission data frames for Bluetooth signal transmission bit allocation examples for whitening care-dedicated control solution information according to the present invention. In the case of the Bluetooth data frame for the LED light patches 203 and 204 attached to the left and right cheeks only for whitening care, bits 5 and 4 are assigned as bits for care time values (levels 1 to 4), and bits 0, 1 and 2 of least significant bits (LSB) as information bits for six cases including dimming levels 1 to 5 for 850 nm LEDs and for off mode selection.

In this case, header and end bytes for Bluetooth communication in FIGS. 8 and 9 are built as BLE Synchronization frames, which is based on a general Bluetooth communication protocol.

As mentioned above, the preferred embodiment of the present invention has been disclosed in the specification and drawings, but persons skilled in the relevant art can appreciate that many modifications and variations are possible in light of the above teachings. It is therefore intended that the scope of the invention be limited not by this detailed description, but rather by the claims appended hereto.

1. A light emitting diode (LED) light patch comprising:
a Bioelectrical Impedance Analysis (BIA) module for sensing the bioelectrical impedance generated by flowing fine current to a stretchable circuit board varied in shape according to contraction and relaxation to thus measure moisture content of a skin surface;
a Bluetooth Low Energy (BLE) module for transmitting the impedance measurement value measured by the BIA module to a user terminal device and receiving the control solution information generated from a skin care service application of the user terminal device;
a battery module having a flexible thin-film battery;
an LED module on which micro-LEDs are mounted in an array;
a power supply circuit having a regulator circuit adapted to supply a given voltage to the respective modules connected to the battery module, a booster circuit adapted to boost the voltage by dimming level, and a battery protection circuit; and
a control processor for performing area-specific control, dimming control, wavelength control and user-selected care time control of the LED module in accordance with the received control solution information,
whereby even if the LED light patch is varied in shape, the power supply state and the bonded states of the circuits are kept stably.

2. The LED light patch according to claim 1, made customizedly and applied to a specific area of facial skin or a skin area of a body and controlled by the skin care service application of the user terminal device.

3. A skin care diagnostic system comprising:
LED light patches each having a BIA module for sensing the bioelectrical impedance to measure moisture content of a skin surface, a BLE module for transmitting the impedance measurement value measured by the BIA module to a user terminal device and receiving the control solution information of the corresponding LED light patch generated from the user terminal device, a battery module having the form of a flexible thin-film battery, an LED module on which micro-LED devices are mounted in an array, and a control processor performing area-specific control, dimming control, wavelength control and user-selected care time control of the LED module in accordance with the received control solution information; and
the user terminal device having a camera module for capturing an image of the skin surface and a skin care service application for diagnosing the images captured by the camera module and the impedance measurement values received from the LED light patches to generate the control solution information of the LED light patches, while interworking with the LED light patches.

4. The skin care diagnostic system according to claim 3, wherein the LED light patches are made customizedly with 8 areas of the facial skin that are made by dividing the entire facial area into a forehead area, both areas around eyes, both cheek areas, both nasolabial fold areas around nose and mouth, and a neck area, while being controlled by the skin care service application of the user terminal device, through independent signals.

5. The skin care diagnostic system according to claim 3, wherein the skin care service application diagnoses skin wrinkle and whitening conditions with levels 1 to 5 and an off mode through the image of the specific area captured by the camera module and diagnoses skin moisture content with grades 1 to 5 and an off mode through the impedance measurement value sensed by the BIA module, so that the skin care service application generates the control solution information for controlling the LED module attached to the specific area.

6. The skin care diagnostic system according to claim 5, wherein the skin care service application interworks with a skin care management server to collect and store the captured skin image and the skin moisture content and thus generate the control solution information for controlling the LED module, while reflecting data of skin conditions of users by age, job, sex, and country.

7. The skin care diagnostic system according to claim 6, wherein the control solution information comprises control information by area for 8 areas of the face, three areas of the forehead, and three areas of the neck, dimming control information for brightness control by diagnosis level, wavelength control information for 630 nm wavelength for wrinkle care and for both of 630 nm and 850 nm wavelengths for whitening care, and care time control information as a user option.

8. A method for operating one or more LED light patches and a skin care diagnostic system using the LED light patches, the method comprising the steps of:
executing a skin care service application embedded in a smartphone to perform Bluetooth interworking with the LED light patches;
capturing images of specific facial areas through a camera module of the smartphone to check the wrinkle and whitening conditions of the specific facial areas;
attaching the LED light patches to the specific facial areas, measuring impedance values of the specific facial areas to check skin moisture content, and transmitting the impedance values to the skin care service application;
analyzing and diagnosing skin conditions through the skin care service application, based on the image and impedance information of the specific facial areas;
generating control solution information of the LED light patches, based on the skin condition diagnosis elements of the specific facial areas analyzed by the skin care service application; and
allowing the LED light patches to receive the control solution information to irradiate light onto the specific facial areas.

9. The method according to claim 8, wherein the step of analyzing and diagnosing skin conditions is performed by allowing the skin care service application to interwork with a skin care management server to collect and store the captured skin images and the skin moisture content and thus generate the control solution information for controlling the LED modules, while reflecting data of skin conditions of users by age, job, sex, and country.

10. The method according to claim 8, wherein the specific facial areas to which the LED light patches are attached are any one or more areas of a forehead area, both areas around eyes, both cheek areas, both nasolabial fold areas around nose and mouth, and a neck area, and the LED patches attached to the specific facial areas are controlled by the skin care service application through independent signals.

11. The method according to claim 8, wherein the control solution information comprises control information by area for 8 areas of the face, three areas of the forehead, and three areas of the neck, dimming control information for brightness control by diagnosis level, and wavelength control information for 630 nm wavelength for wrinkle care and for both of 630 nm and 850 nm wavelengths for whitening care.

12. The method according to claim 11, wherein the control solution information further comprises care time control information as a user option.
